(19) United States
(12) Patent Application Publication
(10) Pub. No.: US 2024/0383855 A1
(43) Pub. Date: Nov. 21, 2024

(54) PREPARATION OF IONIC PHARMACEUTICAL COCRYSTALS USING SOLID AND LIQUID COMPONENTS

(71) Applicant: Texas Tech University System, Lubbock, TX (US)

(72) Inventors: Kristin Hutchins, Lubbock, TX (US); Jesus Daniel Loya, Lubbock, TX (US)

(73) Assignee: Texas Tech University System, Lubbock, TX (US)

(21) Appl. No.: 18/688,767

(22) PCT Filed: Aug. 27, 2022
(86) PCT No.: PCT/US2022/041780

§ 371 Date: Mar. 2, 2024

Related U.S. Application Data
(60) Provisional application No. 63/240,405, filed on Sep. 3, 2021.

(51) Int. Cl. C07D 213/74; C07D 213/38

(52) U.S. Cl. CPC C07D 213/74; C07D 213/38

(57) ABSTRACT

Embodiments of the present disclosure pertain to methods of forming a co-crystallized composition by mixing a first molecule in a solid phase with a second molecule in a liquid phase to form a mixture, and then co-crystallizing the mixture to form the co-crystallized composition in the form of a crystalline solid. The mixing of the first and second molecules may occur through the utilization of mechanical force, such as milling. The co-crystallization of the first and second molecules may occur by adding a solvent to the mixture of the molecules and then evaporating the added solvent. The methods may also include a step of utilizing the co-crystallized composition as seed crystals to grow additional co-crystallized compositions. Further embodiments of the present disclosure pertain to the formed co-crystallized compositions.

### CROSS-REFERENCE TO RELATED APPLICATIONS

This application claims priority to U.S. Provisional Patent Application No. 63/240,405, filed on Sep. 3, 2021. The entirety of the aforementioned patent application is incorporated herein by reference.

### BACKGROUND

Typical co-crystallization methods suffer from numerous drawbacks. For instance, some compounds, such as, but not limited to, oils, chiral compounds, and scarce natural products are difficult to co-crystallize using typical techniques. Numerous embodiments of the present disclosure aim to address the aforementioned limitations.

### SUMMARY

In some embodiments, the present disclosure pertains to methods of forming a co-crystallized composition by mixing a first molecule in a solid phase with a second molecule in a liquid phase to form a mixture. Thereafter, the molecules in the mixture are co-crystallized to form the co-crystallized composition. In some embodiments, the co-crystallized composition is in the form of a crystalline solid.

In some embodiments, the mixing of the first and second molecules occurs through the utilization of mechanical force, such as milling. In some embodiments, the co-crystallization of the first and second molecules occurs by adding a solvent to the mixture of the molecules and then evaporating the added solvent.

In some embodiments, the methods of the present disclosure also include a step of utilizing the co-crystallized composition as seed crystals to grow additional co-crystallized compositions from the first molecule and the second molecule. In some embodiments, the growing includes placing the co-crystallized composition in a solvent that includes a mixture of the first molecule and the second molecule and then evaporating the solvent.

In some embodiments, one of the first molecule or second molecule includes an acidic group capable of donating protons, and the other of the second molecule or first molecule includes a basic group capable of accepting protons. In some embodiments, the acidic group interacts with the basic group to form intermolecular hydrogen bonds in the co-crystallized composition.

Additional embodiments pertain to the co-crystallized compositions of the present disclosure. In some embodiments, the co-crystallized composition includes a first molecule and a second molecule. In some embodiments, the first molecule is co-crystallized in solid phase, and the second molecule is co-crystallized from a liquid phase. In some embodiments, the co-crystallized composition is in the form of a crystalline solid.

### DESCRIPTION OF THE DRAWINGS

FIG. 1A provides an illustration of a method of forming co-crystallized compositions.

FIG. 1B provides a diagram of a method of forming co-crystallized compositions.

FIG. 2 provides chemical structures of several pyridines and bezafibrate (BEZA) with corresponding melting or boiling points.

FIG. 3 provides powder x-ray diffraction (PXRD) patterns of BEZA·3-DMAP (from bottom to top): predicted pattern from X-ray data, crystalline solid obtained from neat milling, slow seeding, fast seeding, and fast precipitation.

FIG. 4 provides a full Fourier transform infrared (FT-IR) spectroscopy spectrum of BEZA·3-DMAP cocrystals, BEZA·3-DMAP crystalline solid after milling, and BEZA·3-DMAP resulting oil after slow evaporation with acetone.

FIG. 5 shows partial FT-IR spectrum highlighting carbonyl region of BEZA·3-DMAP cocrystals, BEZA·3-DMAP crystalline solid after milling, and BEZA·3-DMAP resulting oil after slow evaporation with acetone.

FIGS. 6A-6D show X-ray crystal structures of BEZA·2-MetAP (FIG. 6A), BEZA·2-MetAP highlighting BEZA interactions (FIG. 6B), BEZA·3-MetAP·DCM (FIG. 6C), and BEZA·3-MetAP·DCM showing extended hydrogen-bonding (DCM omitted for clarity) (FIG. 6D).

FIGS. 7A-7D show X-ray crystal structures of BEZA·3-DMAP (FIG. 7A), BEZA·3-PMDA (2:1 form) (FIG. 7B), BEZA·3-PMDA (1:1 form) (FIG. 7C), and BEZA·3-PMDA (1:1 form) highlighting 1D chains of BEZA molecules (FIG. 7D).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Crystal structure determination is an important and widely-used strategy for identification of molecular structures. Crystallization is an important separation and purification technique, and it is a key component of drug development in the pharmaceutical industry. Many organic compounds can be readily crystallized using standard methods. However, some compounds (e.g. oils, chiral compounds, and scarce natural products) are difficult to crystallize using typical techniques.

Co-crystallization has gained considerable attention in the past two decades because it offers a structural characterization strategy. Moreover, co-crystals often exhibit improved physical or chemical properties when compared to the individual component molecules. For instance, mechanochemistry, the use of mechanical force to achieve chemical transformations, has recently re-emerged as a green and efficient method for synthesizing co-crystals. Cryst. Growth Des. 2018, 18, 2495-2501.

Grinding or milling of two solids or a liquid and a solid component have both been used to synthesize cocrystals. Chem. Eur. J. 2008, 14, 747-753; Chem. Commun. 2020, 56, 8293-8296; and Cryst. Growth Des. 2019, 19, 7271-7279. Specifically, for solid-liquid combinations, Jones and coworkers demonstrated neat grinding of the two compounds can afford solid products not accessible from a neat solution growth of the two components. Cryst. Growth Des. 2005, 5, 2233-2241.

The crystal structures of many organic compounds can be determined by growing single crystals using standard laboratory methods and collecting X-ray data using single-crystal X-ray diffraction. However, some organic compounds (e.g., oils, chiral compounds, and scarce natural products) prove challenging to crystallize using standard methods.

Accordingly, a need exists for more effective co-crystallization compositions and methods for forming co-crystallized compositions. Various embodiments of the present disclosure address the aforementioned need.

In some embodiments, the present disclosure pertains to methods of forming co-crystallized compositions. In some embodiments, the method generally includes one or more of the following steps of: (1) mixing a first molecule with a second molecule to form a mixture; and (2) co-crystallizing the first molecule with the second molecule to form the co-crystallized composition. In some embodiments, the first molecule is in a solid phase. In some embodiments, the second molecule is in a liquid phase. In some embodiments, the co-crystallized composition is in the form of a crystalline solid.

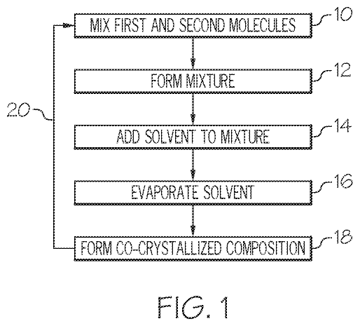

In some embodiments illustrated in FIG. 1A, the co-crystallization methods of the present disclosure include mixing a first molecule with a second molecule (step 10) to form a mixture (step 12); adding a solvent to the mixture (step 14); and evaporating the solvent (step 16) to form the co-crystallized composition (step 18). In some embodiments, the methods of the present disclosure also include a step of utilizing the co-crystallized composition as seed crystals for seed growth of additional co-crystallized compositions from the first molecule and the second molecule (step 20). FIG. 1B illustrates a more specific co-crystallization method according to an embodiment of the present disclosure.

Additional embodiments of the present disclosure pertain to co-crystallized compositions. In some embodiments, the co-crystallized compositions include a first molecule and a second molecule. In some embodiments, the first molecule is co-crystallized from a solid phase. In some embodiments, the second molecule is co-crystallized from a liquid phase. In some embodiments, the co-crystallized composition is in the form of a crystalline solid. In some embodiments, the co-crystallized composition is formed in accordance with the methods of the present disclosure.

### Method of Forming a Co-Crystallized Composition

As set forth in further detail herein, an aspect of the present disclosure pertains to a method of forming a co-crystallized composition. In some embodiments, the method generally includes one or more of the following steps of: (1) mixing a first molecule with a second molecule to form a mixture; and (2) co-crystallizing the first molecule with the second molecule to form the co-crystallized composition. In some embodiments, the first molecule is in a solid phase. In some embodiments, the second molecule is in a liquid phase. In some embodiments, the co-crystallized composition is in the form of a crystalline solid.

As outlined in further detail herein, the methods of the present disclosure can include various mechanisms for mixing and co-crystallizing. Furthermore, the first molecule in the solid phase, the second molecule in the liquid phase, and the co-crystallized compositions can have numerous embodiments. In addition, the methods of the present disclosure can include additional steps, such as, but not limited to, seed growth.

### Mixing

As set forth in further detail herein, various mechanisms of mixing can be utilized to form the co-crystallized compositions of the present disclosure. For instance, in some embodiments, the mixing occurs through the utilization of a mechanical force. In some embodiments, the mechanical force includes milling. In some embodiments, the milling includes ball milling. In some embodiments, the milling occurs through the utilization of a shaker mill, a mortar pestle, a planetary mill, a speed mixer, or combinations thereof.

Mixing can occur for various periods of time. For instance, in some embodiments, the mixing takes place for at least 30 seconds. In some embodiments, the mixing takes place for at least 60 seconds. In some embodiments, the mixing takes place for at least 2 minutes. In some embodiments, the mixing takes place for at least 3 minutes. In some embodiments, the mixing takes place for at least 5 minutes. In some embodiments, the mixing takes place for at least 30 minutes. In some embodiments, the mixing takes place for at least 1 hour.

### Co-Crystallizing

As detailed herein, various methods of co-crystallization can be utilized in the methods of the present disclosure. For example, in some embodiments, the co-crystallizing includes adding a solvent to the mixture and then evaporating the added solvent. In some embodiments, the mixture is in the form of a liquid (e.g., an oil) at the time of adding the solvent.

In some embodiments, a solvent may be added to a mixture during a mixing step. In some embodiments, a solvent may be added to a mixture after a mixing step.

Various solvents may be added to the mixtures of the present disclosure. For instance, in some embodiments, the solvent includes an organic solvent. In some embodiments, the organic solvent includes, without limitation, acetonitrile, alcohols (e.g., ethanol, methanol, isopropanol), acetone, toluene, ethyl acetate, dichloromethane, tetrahydrofuran, cyclohexane, N,N′-dimethylformamide, chloroform, or combinations thereof. In some embodiments, the solvent includes an aqueous solvent. In some embodiments, the aqueous solvent includes water.

In some embodiments, the evaporating occurs gradually through time. In some embodiments, the gradual evaporation occurs during a period of at least 6 hours. In some embodiments, the gradual evaporation occurs during a period of at least 12 hours. In some embodiments, the gradual evaporation occurs during a period of at least 24 hours. In some embodiments, the gradual evaporation occurs during a period of at least 48 hours. In some embodiments, the gradual evaporation occurs during a period of at least 72 hours.

Evaporation can occur through numerous mechanisms. For instance, in some embodiments, the evaporating occurs by vaporization.

### First Molecules

In the present disclosure, first molecules generally refer to molecules that are in a solid phase. As set forth in further detail herein, the first molecules of the present disclosure can have numerous embodiments and be in various solid phases. For instance, in some embodiments, the first molecule includes an acidic group capable of donating protons. In some embodiments, the acidic group includes carboxylic acid.

In some embodiments, the first molecule includes a basic group capable of accepting protons. In some embodiments, the basic group includes an amine group. In some embodiments, the amine group includes an amino pyridine. In some embodiments, the amino pyridine includes, without limitation, 2-(methylamino)pyridine, 3-(methylamino)pyridine, 3-dimethylaminopyridine, 3-pyridinemethyldimethyl amine, and combinations thereof.

In some embodiments, the first molecule is a drug. In some embodiments, the drug is a polymorphic drug. In some embodiments, the first molecule is a drug that can include, without limitation, anti-inflammatory drugs, anti-cholesterol drugs, ritonavir, carbamazepine, cimetidine, propranolol, atenolol, labetalol, metoprolol, acebutolol, bezafibrate, naproxen, mefenamic acid, diclofenac, acetazolamide, furosemide, aceclofenac, vitamin B3 , beta blockers, chiral compounds, derivatives thereof, and combinations thereof.

In some embodiments, the first molecule is bezafibrate. In some embodiments, the first molecule is a beta blocker. In some embodiments, the beta blocker includes, without limitation, atenolol, bisoprolol, carvedilol, labetalol, metoprolol, propranolol, sotalol, acebutolol, and combinations thereof.

In some embodiments, the first molecules of the present disclosure can be in a solid phase at various temperatures. For instance, in some embodiments, the first molecules are in a solid phase at room temperature.

### Second Molecules

In the present disclosure, second molecules generally refer to molecules that are in a liquid phase. As detailed herein, the second molecules of the present disclosure can have numerous embodiments and be in various liquid phases.

In some embodiments, the second molecule includes an acidic group capable of donating protons. In some embodiments, the acidic group includes carboxylic acid.

In some embodiments, the second molecule includes a basic group capable of accepting protons. In some embodiments, the basic group includes an amine group. In some embodiments, the amine group includes a pyridine-based compound. In some embodiments, the pyridine-based compound includes an amino pyridine. In some embodiments, the amino pyridine includes, without limitation, 2-(methylamino)pyridine, 3-(methylamino)pyridine, 3-dimethylaminopyridine, 3-pyridinemethyldimethyl amine, and combinations thereof.

In some embodiments, the second molecules of the present disclosure can be in a liquid phase at various temperatures. For instance, in some embodiments, the second molecule is in the liquid phase at room temperature.

In some embodiments, the second molecules of the present disclosure can be in various forms of a liquid phase. For instance, in some embodiments, the second molecule in the liquid phase is a viscous liquid at room temperature. In some embodiments, the second molecule in the liquid phase is in the form of oils at room temperature.

The first molecules and second molecules of the present disclosure can have various molecular weights. For instance, in some embodiments, at least one of the first molecule or second molecule has a molecular weight of more than 200 grams per mole. In some embodiments, at least one of the first molecule or second molecule has a molecular weight of more than 250 grams per mole. In some embodiments, the first molecule has a molecular weight of more than 200 grams per mole. In some embodiments, the first molecule has a molecular weight of more than 250 grams per moles.

### Co-Crystallized Composition

As outlined herein, the co-crystallized compositions formed via the methods of the present disclosure can have numerous embodiments and be in various forms. For instance, in some embodiments, the co-crystallized composition is in an ionic form. In some embodiments, the co-crystallized composition is in a partially ionic form. In some embodiments, the co-crystallized composition is in neutral form. In some embodiments, the co-crystallized composition includes ionic co-crystals of the first molecule and the second molecule. In some embodiments, the first molecule of the co-crystallized composition is in anionic form. In some embodiments, the second molecule of the co-crystallized composition is in cationic form.

In some embodiments, one of the first molecule or second molecule includes an acidic group capable of donating protons, and the other of the second molecule or first molecule includes a basic group capable of accepting protons. In some embodiments, the acidic group interacts with the basic group to form intermolecular hydrogen bonds in the co-crystallized composition.

In some embodiments, the first molecule in the co-crystallized composition includes an acidic group capable of donating protons, and the second molecule in the co-crystallized composition includes a basic group capable of accepting protons. In some embodiments, the acidic group of the first molecule interacts with the basic group of the second molecule to form intermolecular hydrogen bonds.

In some embodiments, the second molecule in the co-crystallized composition includes an acidic group capable of donating protons, and the first molecule in the co-crystallized composition includes a basic group capable of accepting protons. In some embodiments, the acidic group of the second molecule interacts with the basic group of the first molecule to form intermolecular hydrogen bonds.

In some embodiments, the acidic group interacts with the basic group to form intermolecular bonds. In some embodiments, the intermolecular bonds lack halogen bonds. In some embodiments, the basic group and the acidic group in the co-crystallized compositions lack halogens.

The co-crystallized compositions of the present disclosure can represent various types of compositions. For instance, in some embodiments, the co-crystallized composition represents a pharmaceutical composition.

### Co-Crystal Growth

As detailed herein, the methods of forming co-crystallized compositions of the present disclosure can include additional steps. For instance, in some embodiments, the methods of the present disclosure can further include a step of utilizing the co-crystallized composition as seed crystals to grow additional co-crystallized compositions from the first molecule and the second molecule.

In some embodiments, the growing includes placing the co-crystallized composition in a solvent having a mixture of the first molecule and the second molecule, and evaporating the solvent. In some embodiments, the evaporating occurs gradually. In some embodiments, the evaporating occurs rapidly.

In some embodiments, the solvents include organic solvents. In some embodiments, the organic solvents include, without limitation, acetonitrile, alcohols (e.g., ethanol, methanol, isopropanol), acetone, toluene, ethyl acetate, dichloromethane, tetrahydrofuran, cyclohexane, N,N′-dimethylformamide, chloroform, and combinations thereof.

### Additional Embodiments

Reference will now be made to more specific embodiments of the present disclosure and experimental results that provide support for such embodiments. However, Applicant notes that the disclosure below is for illustrative purposes only and is not intended to limit the scope of the claimed subject matter in any way.

### EXAMPLE 1. MECHANOCHEMISTRY AS A TOOL FOR CRYSTALLIZING INACCESSIBLE SOLIDS FROM VISCOUS LIQUID COMPONENTS

In this Example, Applicant describes a strategy for crystallizing liquids using a solid component that facilitates crystallization of the components into a multi-component solid. The method utilizes mechanochemistry as a key step in synthesis of the crystals, which are otherwise difficult or impossible to obtain using standard solution-growth techniques. Spectroscopic characterization demonstrates a difference in products obtained from mechanochemistry and failed solution crystallizations. The crystals obtained using the method can also be used to seed subsequent crystallizations in solution when the solids are not obtainable without seed crystals. In addition to obtaining the first X-ray structures including three common pyridine ligands, the methods described provide a useful strategy for obtaining crystalline materials when the components do not crystallize using standard solution conditions or are viscous liquids or oils at room temperature.

In this Example, Applicant chose to use a solid compound that would interact with the liquids through intermolecular hydrogen bonds to facilitate crystallization of the liquid. However, co-crystallization using slow evaporation of the two components in a neat solution or with a solvent yielded ionic cocrystals in some cases, but more frequently afforded oils or single crystals of the pure solid component.

Applicant implemented mechanochemistry to enhance molecular diffusion and increase reactivity of the molecules toward cocrystal formation. Neat milling of the liquid and solid followed by dissolution of the milled product in an organic solvent yields single crystals suitable for X-ray diffraction (FIG. 1B). Applicant further shows that crystals obtained by milling and dissolution can be used to synthesize bulk solids via seeding.

Grinding of two solids followed by dissolution to yield co-crystals is common. However, to Applicant's knowledge, there have not been reports that use milling of a liquid and solid followed by dissolution of the resultant oil to form single cocrystals. Applicant expects the method to be useful for crystallizing liquids, oils, or natural products.

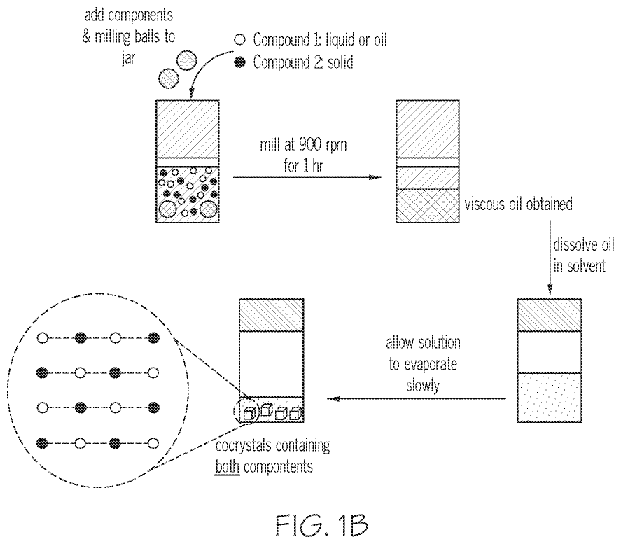

The liquids Applicant selected for crystallization include four pyridine-based compounds, namely, 2-(methylamino)pyridine (2-MetAP), 3-(methylamino)pyridine (3-MetAP), 3-dimethylaminopyridine (3-DMAP) and 3-pyridinylmethyldimethyl amine (3-PMDA) (FIG. 2). 2-MetAP, 3-MetAP, and 3-DMAP have been utilized as ligands in metal-organic materials to capture CO₂ and perform catalysis. These aminopyridine liquids are similar in viscosity to the analogous methylpyridine compounds. Only 2-MetAP has been incorporated into solid forms and characterized structurally (single component, cocrystals, and metal-organic solids). The solid-state structures of 3-MetAP, 3-DMAP, and 3-PMDA are unknown.

To aid in crystallization of the liquids, Applicant selected bezafibrate (BEZA), an anti-cholesterol drug, as a solid co-former. Applicant selected BEZA for three reasons: 1) it contains an acid group, which could facilitate co-crystallization with the liquid pyridines through hydrogen bonding; 2) BEZA often crystallizes as a single-component solid and identification of single BEZA crystals are facile; and 3) Applicant's ongoing interest in the drug.

### Example 1.1. Method Development

Co-crystallization experiments with the four liquid pyridine compounds 2-MetAP, 3-MetAP, 3-DMAP, or 3-PMDA and BEZA using a conventional method of slow solvent evaporation was attempted first. Both components were dissolved in an organic solvent, and the solution was left to evaporate slowly. Co-crystallization attempts using slow evaporation yielded oils, even when the solvents and ratios were altered. Solvent layering methods were attempted, but experiments also yielded oily, non-crystalline substances. If higher concentrations of BEZA were used and exceeded the concentration of the pyridine, thin needles of pure BEZA were obtained.

Applicant encountered one exception in the solution-based co-crystallization attempts. The solid BEZA·2-MetAP was successfully synthesized by slow evaporation using one solvent (acetonitrile). Applicant also attempted to grow cocrystals by dissolving BEZA in the neat liquid pyridines because the solubility was good and use of an organic solvent could be avoided. With 2-MetAP and excess 3-DMAP, Applicant obtained co-crystalline solids. However, in the other two cases, Applicant obtained oils or single crystals of pure BEZA.

Thus, Applicant sought an alternative method to reliably crystallize the liquids via co-crystallization methods and turned to mechanochemistry. The liquid and solid component were combined in a milling jar and subjected to mechanochemical milling using a commercial ball mill. Neat milling of solid BEZA and the liquid pyridine 3-MetAP, 3-DMAP, or 3-PMDA afforded a viscous oil or crystalline solid (Table 1).

TABLE 1
Summary of the products obtained from solution, milling, and seeding experiments.
An identical phase means that the PXRD pattern of the product showed good correlation
with the simulated pattern from SCXRD data. A non-identical phase means that the
PXRD pattern of the product did not match the simulated pattern from SCXRD data.

| | | Slow | | Neat | | | |
|---|---|---|---|---|---|---|---|
| | | evaporation | | milling + | | | |
| | Slow | using neat | Neat | slow | Fast | Slow | Fast |
| Crystal | evaporation | pyridine | milling | evaporation | precipitation | Seeding | seeding |
| BEZA•2- | salt- | salt- | Oil | oil | crystalline | crystalline | n/a |
| MetAP | cocrystal | cocrystal | | | solid | solid | |
| | | | | | (identical | (identical | |
| | | | | | phase) | phase) | |
| BEZA•3- | oil | oil | oil | salt-solvate | crystalline | crystalline | crystalline |
| MetAP• | | | | | solid | solid | solid |
| DCM | | | | | (non-identical | (identical | (identical |
| | | | | | phase) | phase) | phase) |
| BEZA•3- | oil | salt- | crystalline | salt- | crystalline | crystalline | crystalline |
| DMAP | | cocrystal | solid | cocrystal | solid | solid | solid |
| | | | | | (identical | (identical | (identical |
| | | | | | phase) | phase) | phase) |
| BEZA•3- | oil | oil | oil | salt- | crystalline | salt | crystalline |
| PMDA | | | | cocrystal | solid | (1:1 form) | solid |
| | | | | (2:1 form) | (non-identical | | (mixture |
| | | | | | phase) | | of 2:1 and |
| | | | | | | | 1:1 forms) |

To obtain single cocrystals, the product obtained from neat milling was subsequently dissolved in an organic solvent, the solution was allowed to evaporate slowly, and single crystals large enough for single-crystal X-ray diffraction (SCXRD) were obtained. This method yielded ionic or partially ionic cocrystals of BEZA·3-MetAP·DCM (solvate with dichloromethane (DCM)), BEZA·3-DMAP, and BEZA·3-PMDA (2:1 form).

All the obtained solids are ionic or partially ionic in nature. If the solid is fully ionic, it is classified as an ionic cocrystal or a salt. If the solid is partially ionic, it is classified as a salt-cocrystal. The formulas of all solids were confirmed by SCXRD and ¹H NMR spectroscopy. Powder X-ray diffraction (PXRD) and FT-IR spectroscopy were used to characterize oils and bulk crystalline solids.

In addition to the slow evaporation and milling experiments outlined in this Example, several other experiment types were conducted in efforts to synthesize multi-component solids incorporating the liquid component and to compare the mechanochemical method to other traditional solution methods (Table 1). These experiments included: (1) fast precipitation using simple solutions, (2) allowing the resulting oil from neat milling to sit undisturbed for several months to determine if single crystals would grow from the oil, and (3) seeding crystal growth using single crystals obtained from neat milling and subsequent slow evaporation as seeds in fresh solution cocrystallizations.

### Example 1.2. Fast Precipitation

Fast precipitation experiments using simple solutions of the pyridine and BEZA in an organic solvent were conducted. High concentrations of the two components were combined in a minimal amount of organic solvent, and crystallization was allowed to occur in solution over 1-2 days. All experiments yielded microcrystalline solids. However, the crystals were not large enough for SCXRD and were, thus, characterized by PXRD.

Two of the crystalline solids, BEZA·2-MetAP and BEZA·3-DMAP, showed good correlation with the predicted PXRD patterns based on single-crystal data. On the other hand, fast precipitation of BEZA with 3-MetAP or 3-PMDA afforded crystalline solids, but the PXRD patterns did not show good correlation with the predicted patterns from single-crystal data. The patterns did not match that of pure BEZA.

### Example 1.3. Neat Milling

Neat milling of the pyridines with BEZA afforded oils in three of the four cases (with 2-MetAP, 3-MetAP, and 3-PMDA). Directly following the neat milling experiments, PXRD characterization was performed for each oil by removing the oil from the milling jar, placing it on a watch glass, and then transferring the oil to the PXRD sample holder. PXRD demonstrated that each oil exhibited crystallinity. FT-IR spectroscopy for each oil was also performed.

The PXRD pattern of the oil obtained from milling BEZA with 2-MetAP showed good correlation to the simulated pattern from SCXRD data. The FT-IR spectrum of the oil from milling also showed excellent correlation with the spectrum of the salt-cocrystals. Thus, milling facilitates direct cocrystallization of 2-MetAP and BEZA.

On the other hand, the PXRD pattern of the oil obtained from milling BEZA with 3-MetAP showed poor correlation to the simulated PXRD pattern from SCXRD data. The FT-IR spectrum of the oil showed partial overlap with the spectrum of the crystals, but some signals were shifted or absent. In this case, milling aids in co-crystallization but does not afford salt-solvate crystals directly.

The PXRD pattern of the oil obtained from milling BEZA with 3-PMDA surprisingly gave evidence of a new crystal form, which Applicant subsequently determined to be the 1:1 form (see seeding Example 1.4). The FT-IR spectrum of the oil after milling also corroborated this observation because it showed excellent correlation with the 1:1 salt spectrum.

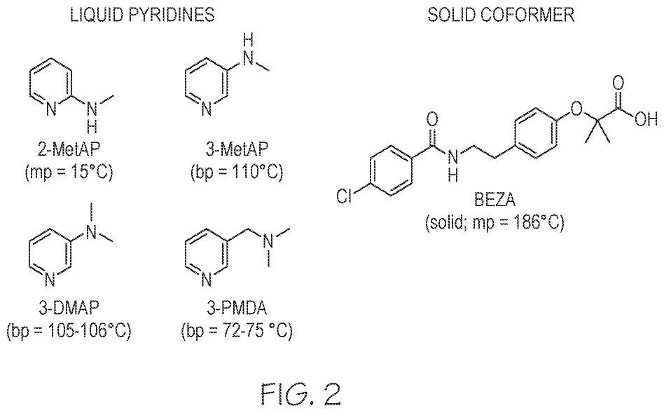

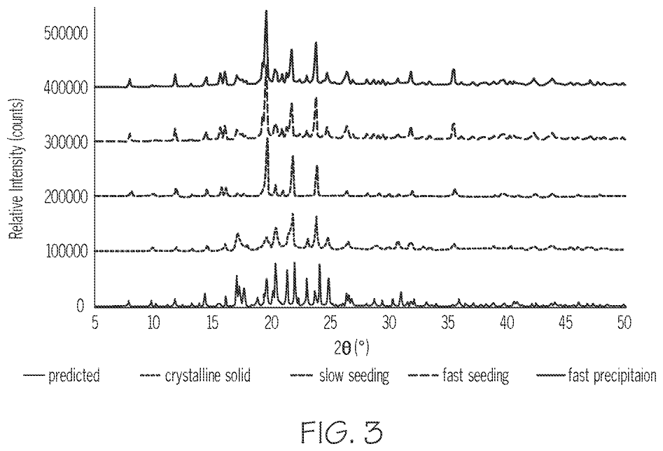

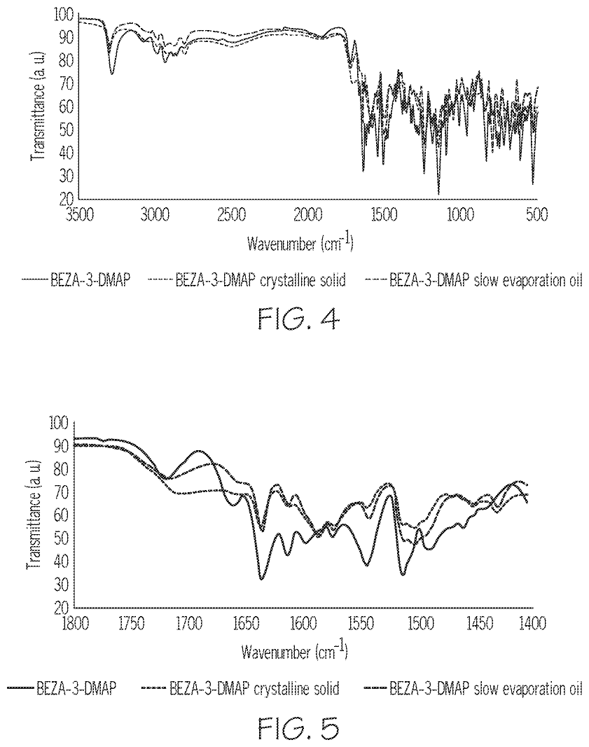

Akin to numerous other literature examples of milling solid and liquid components, neat milling of solid BEZA and liquid 3-DMAP afforded a crystalline solid rather than an oil as in the other three cases. The PXRD pattern showed good correlation to the simulated pattern based on SCXRD data (FIG. 3). Moreover, the FT-IR spectrum of the solid after milling showed excellent correlation to the salt-cocrystals (FIGS. 4-5). Thus, milling also facilitates direct co-crystallization of 3-DMAP and BEZA.

The products obtained from neat milling experiments were allowed to sit undisturbed for several (eight to eleven) months. Initially, Applicant observed oils for BEZA·3-MetAP and BEZA·3-PMDA, and a crystalline solid for BEZA·3-DMAP. After several months, the oils and crystalline solid appeared completely dried. Initially, optical imaging demonstrated small crystals in BEZA·3-MetAP, a grainy solid for BEZA·3-DMAP, and some solid pieces for BEZA·3-PMDA. After several months, small crystals were observed for BEZA·3-MetAP and BEZA·3-DMAP, while BEZA·3-PMDA appeared to look the same when it was first imaged. However, none of the crystals were large enough for SCXRD studies. The oils from solution growth did not show evidence of spontaneous crystallization.

### Example 1.4. Seeding

Seeding experiments were used to determine if crystals obtained from milling and subsequent slow evaporation could be used as seeds in fresh solution co-crystallizations to synthesize bulk solids. Slow seeding was performed by making a solution containing a low concentration of BEZA (25-75 mg) and the pyridine in the same molar ratio the ionic cocrystal was synthesized. After the solution was made, a single ionic cocrystal synthesized using the new technique (milling and subsequent slow evaporation) was dropped in the solution, and the solution was allowed to evaporate slowly for 2-3 days. After a period of 2-3 days, the solvent had evaporated and small, thin needles formed. Fast seeding was performed by making a solution containing a higher concentration of BEZA (50-150 mg) and the pyridine in the same molar ratio the ionic cocrystal was synthesized. After the solution was made, a single ionic cocrystal synthesized from the new technique was dropped in the solution, and the solution was allowed to evaporate slowly for 3-4 days. After a period of 1-2 days, small, thin needles formed and after 3-4 days the solvent had evaporated. PXRD data was collected for each slow and fast seeding sample.

The seeding experiments proved to be successful for BEZA·2-MetAP, BEZA·3-MetAP·DCM, and BEZA·3-DMAP. PXRD patterns of the crystalline solids obtained from seeding showed good correlation to the simulated PXRD patterns based on SCXRD data.

Although suitable multi-component crystals cannot be reproducibly obtained using solution methods initially, ionic cocrystals obtained using mechanochemistry followed by slow evaporation can be used to seed co-crystallization from fresh solutions and afford bulk samples. The single crystal obtained through the milling and subsequent slow evaporation process acts a nucleation site and decreases the time of crystal growth when added to the solution. In a typical solution without a seed, crystal growth often occurs from multiple nucleation sites. The seed crystal used in this Example provides a nucleation site and directs formation of the multi-component solid.

As mentioned in Example 1.3, neat milling of 3-PMDA and BEZA afforded an oil, and subsequent solution growth yielded the 2:1 salt-cocrystal. However, the PXRD pattern of the oil did not correlate with the 2:1 form that was obtained. Slow seeding experiments with 3-PMDA and BEZA afforded a new crystal form (as evidenced by PXRD), and some of the crystals were suitable for SCXRD studies. The crystal form obtained by slow seeding includes BEZA and 3-PMDA in a 1:1 ratio, which is a stoichiometric polymorph of the 2:1 form. Fast seeding experiments with BEZA and 3-PMDA afforded a mixture of the two forms, as evidenced by PXRD. Re-examination of the PXRD pattern of the oil obtained from neat milling shows good correlation with the 1:1 form.

Applicant's initial milling and slow evaporation experiment did afford the 2:1 form of BEZA·3-PMDA. However, since the initial experiment, Applicant have only been able to synthesize the 1:1 form of BEZA·3-PMDA. The fact that slow seeding and the oil obtained from neat milling afford the 1:1 form, while fast precipitation gives a mixture of the 2:1 and 1:1 forms points to the possibility that the 2:1 form is a kinetic product, while the 1:1 form is the thermodynamically favored form.

### Example 1.5. X-Ray Crystal Structures

Although the components in the ionic cocrystals are structurally simple, the crystal structures are surprisingly complex. As expected, the carboxylic acid of BEZA engages in intermolecular hydrogen bonds with the pyridine molecules in the solid state, but the interaction does not always occur at the pyridyl nitrogen. Since these are the first X-ray structures including the liquids 3-MetAP, 3-DMAP, and 3-PMDA, the structures are described in detail herein.

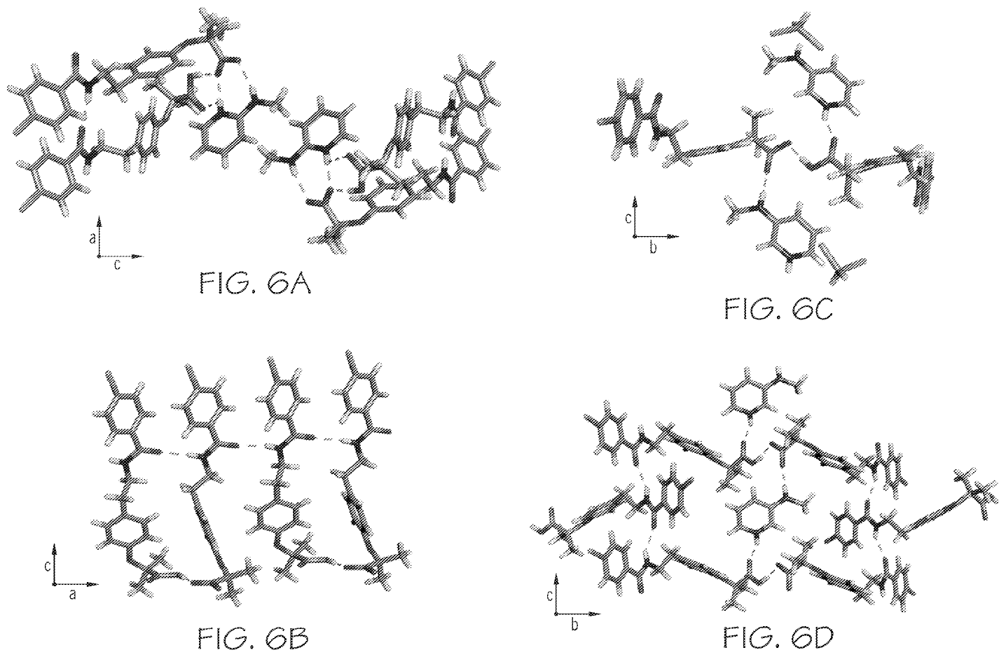

BEZA and 2-MetAP crystallized in a 2:1 ratio as a salt-cocrystal in the triclinic space group, P1̅. The asymmetric unit of BEZA·2-MetAP contains four molecules of BEZA and two molecules of 2-MetAP. Both 2-MetAP molecules are protonated at the pyridine position. Two of the four unique BEZA molecules contain neutral carboxylic acid groups and two contain deprotonated carboxylic acids. Deprotonated BEZA molecules engage with molecules of 2-MetAP via a two-point interaction that includes O⁻ . . . H—N⁺ and N—H . . . O hydrogen bonds (FIG. 6A).

On the other hand, protonated BEZA molecules only interact with other BEZA molecules through O—H . . . O hydrogen bonds. Additionally, the BEZA molecules self-assemble into one-dimensional (1D) hydrogen-bonded chains through N—H . . . O hydrogen bonds between amide groups (FIG. 6B).

BEZA and 3-MetAP crystallized in a 1:1 ratio as a salt-solvate with DCM included in the lattice. The solvate BEZA·3-MetAP·DCM includes one molecule of each type in the asymmetric unit, and the components crystallized in the monoclinic space group P2₁/c. The DCM solvent molecule interacts with BEZA via C—H . . . O hydrogen bonds. The pyridine of 3-MetAP engages in a charge-assisted N⁺—H . . . O⁻ hydrogen bond with BEZA, and the amine of 3-MetAP engages in a N—H . . . O hydrogen bond with the carboxylate of BEZA (FIGS. 6C-6D).

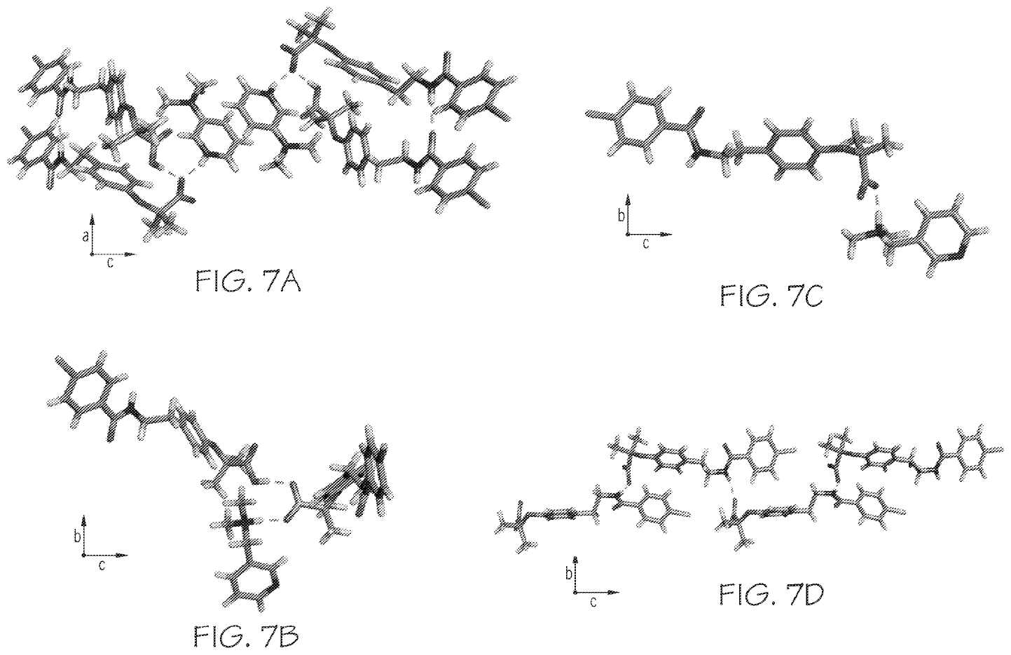

BEZA and the pyridine 3-DMAP crystallized in a 2:1 ratio as a salt-cocrystal in the triclinic space group, P1̅. The structure of BEZA·3-DMAP is nearly isostructural to BEZA·2-MetAP. However, 3-DMAP lacks a hydrogen-bond donor amine group in the 2-position. The asymmetric unit of BEZA·3-DMAP contains four BEZA molecules and two protonated 3-DMAP molecules. The deprotonated BEZA molecules engage with 3-DMAP molecules via charge-assisted O⁻ . . . H—N⁺ hydrogen bonds (FIG. 7A). The BEZA molecules engage in O—H . . . O hydrogen bonds between protonated carboxylic acids and deprotonated carboxylates.

The pyridine 3-PMDA differs from the other pyridine molecules used in this study because there is an extra CH₂ group between the ring and the amine (FIG. 2). Following the first neat milling and slow evaporation experiment, Applicant obtained the 2:1 form of BEZA·3-PMDA, which is a salt-cocrystal. The components of BEZA·3-PMDA (2:1) crystallized in the monoclinic space group, P2₁/n. The asymmetric unit of BEZA·3-PMDA (2:1) contains one protonated molecule of 3-PMDA and two crystallographically unique BEZA molecules, one protonated and one deprotonated. The 3-PMDA molecule is protonated at the amine nitrogen, rather than the pyridyl nitrogen as in all the previous cases. 3-PMDA interacts with the deprotonated BEZA though charge-assisted hydrogen bonds at the amine position (FIG. 7B). The neutral pyridine nitrogen of 3-PMDA engages in C—H . . . N interactions with a methyl group of BEZA.

Applicant obtained a second form of BEZA·3-PMDA (1:1) via seeding experiments. BEZA·3-PMDA (1:1) is a salt, and the components crystallized in the orthorhombic space group Pbca. The asymmetric unit of BEZA·3-PMDA (1:1) contains one deprotonated molecule of BEZA and one protonated molecule of 3-PMDA. Identical to the 2:1 form, the amine of 3-PMDA is protonated rather than the pyridine. Recognition between 3-PMDA and BEZA occurs via a charge-assisted N⁺—H . . . O hydrogen bond between the amine of 3-PMDA and the carboxylate of BEZA (FIG. 7C). The pyridyl nitrogen of 3-PMDA engages in C—H . . . N hydrogen bonds with the halogenated ring of BEZA. The amide N—H group of BEZA interacts with the second oxygen of the carboxylate of BEZA via N—H . . . O⁻ hydrogen bonds to form 1D chains (FIG. 7D).

### Example 1.6. Mechanism Behind Cocrystal Formation

Understanding why the technique of neat milling followed by dissolution allows formation of ionic cocrystals inaccessible by other methods is important for this work. The intermolecular interactions that facilitate co-crystallization of the pyridines are strong, and, in fact, all the synthesized cocrystals are ionic or partially ionic in nature. The pKₐ rule is widely used for the design of salts and cocrystals, especially within the pharmaceutical industry. Applicant calculated pKₐ values for each liquid pyridine and BEZA. For crystallizations of 2-MetAP, 3-MetAP, and 3-DMAP with BEZA, the difference between the pKₐ values of the protonated base and acid (ΔpKₐ) falls in the salt-cocrystal continuum with the probability of proton transfer at >58%. Indeed, salts did form in all three cases. With both stoichiometric forms of BEZA·3-PMDA, BEZA engages in hydrogen bonds at the amine position of 3-PMDA. The pKₐ value for the pyridyl nitrogen is quite low compared to the other three pyridines studied here, which explains why protonation occurs at the amine. The ΔpKₐ calculation for BEZA and 3-PMDA (using the pKₐ of the protonated amine) indicates salt formation is expected, and this was indeed observed in both stoichiometric forms. FT-IR spectroscopy of the cocrystals demonstrated a noticeable shift in the carbonyl region when comparing neutral BEZA (C═O around 1700 cm⁻¹) to the deprotonated form observed in the ionic cocrystals (carboxylate ˜1640 cm⁻¹).

In this Example, milling facilitates co-crystallization of the liquid pyridines either directly or following dissolution in a solvent. Friščić and Jones stated that “cocrystallization by neat grinding a liquid reactant (i.e., no additional liquid) is different to cocrystal formation by recrystallizing a solid cocrystal component the same liquid reactant.” Cryst. Growth Des. 2009, 9, 1621-1637. Here, neat dissolution (or recrystallizing a solid in the liquid reactant) did yield cocrystals in two cases, BEZA·2-MetAP and BEZA·3-DMAP, while for 3-MetAP and 3-PMDA, only oils and single crystals of pure BEZA were obtained.

Using FT-IR spectroscopy, Applicant compared the spectra of the cocrystals, the product following neat milling, and the oil obtained from slow solvent evaporation attempts using an organic solvent. In the case of 2-MetAP, the spectrum of the oil from solvent evaporation (using a solvent other than acetonitrile) shows broadening and some signals are absent compared to the salt-cocrystals and product from milling. For 3-DMAP, the spectrum of the oil from solvent evaporation also shows some broadening, but it correlates well with the salt-cocrystals and product from milling. In the case of 3-MetAP, the spectrum of the oil from solvent evaporation shows excellent correlation with the product from milling, but both spectra show broadening and missing signals when compared to the salt-solvate spectrum.

Based on the aforementioned results, Applicant believes that milling facilitates ionic cocrystal formation and the milled product contains small particles of ionic cocrystals. Upon using an appropriate solvent following milling, single ionic cocrystals can be formed. This is clearly apparent with 2-MetAP, 3-MetAP, and 3-DMAP. For 2-MetAP, the oil from slow evaporation differs spectroscopically from the salt-cocrystal and milling product. For 3-MetAP, the salt structure includes a DCM molecule. Milling followed by growth from DCM yields salt-solvate crystals, whereas solution growth from DCM directly did not. For 3-DMAP, the solid from milling shows excellent correlation to the salt-cocrystals. Initial solution growth did not afford single crystals even though the IR spectrum correlated well. In the case of 3-PMDA, milling was also necessary for co-crystallization, and we identified two stoichiometric polymorphs, wherein the 1:1 form is more favored.

### Example 1.7. Conclusions

Here, Applicant demonstrated that mechanochemical co-crystallization is a viable method for facilitating crystallization of liquids. The method is facile and includes neat mechanochemical milling of the components, dissolution of the milling product into a solvent, and slow evaporation to yield single crystals suitable for SCXRD. Applicant envisions that this method will be especially useful in synthesizing single crystals and bulk crystalline solids that are difficult to obtain using conventional crystallization methods and for compounds that are oils or liquids at room temperature. Importantly, the single crystals obtained using the method can be used as seeds to synthesize the desired solids in bulk, when such solids are inaccessible by using traditional solution-growth methods.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of forming a co-crystallized composition, said method comprising:
mixing a first molecule with a second molecule to form a mixture,
wherein the first molecule is in a solid phase, and
wherein the second molecule is in a liquid phase;
co-crystallizing the first molecule with the second molecule to form the co-crystallized composition,
wherein the co-crystallized composition is in the form of a crystalline solid.

2. The method of claim 1, wherein the mixing comprises milling.

3. (canceled)

4. The method of claim 1, wherein the co-crystallizing comprises adding an organic solvent to the mixture and evaporating the added solvent, wherein the mixture is in the form of a liquid at the time of adding the solvent, and wherein the solvent is added after the mixing.

5-7. (canceled)

8. The method of claim 4, wherein the organic solvent is selected from the group consisting of acetonitrile, alcohols, acetone, toluene, ethyl acetate, dichloromethane, tetrahydrofuran, cyclohexane, N,N′-dimethylformamide, chloroform, and combinations thereof.

9. The method of claim 4, wherein the evaporating occurs during a period of at least 6 hours, and wherein the evaporating occurs by vaporization.

10. (canceled)

11. The method of claim 1, wherein one of the first molecule or second molecule comprises an acidic group capable of donating protons, and wherein the other of the first molecule or second molecule comprises a basic group capable of accepting protons.

12. The method of claim 11, wherein the acidic group interacts with the basic group to form intermolecular hydrogen bonds in the co-crystallized composition.

13. The method of claim 11, wherein the acidic group interacts with the basic group to form intermolecular bonds, wherein the intermolecular bonds lack halogen bonds.

14. The method of claim 11, wherein the basic group and the acidic group lack halogens.

15. The method of claim 11, wherein the first molecule comprises an acidic group capable of donating protons, and wherein the second molecule comprises a basic group capable of accepting protons.

16. The method of claim 11, wherein the first molecule comprises a basic group capable of donating protons, and wherein the second molecule comprises an acidic group capable of accepting protons.

17. The method of claim 1, wherein the acidic group comprises carboxylic acid, and wherein the basic group comprises an amine group.

18. (canceled)

19. The method of claim 17, wherein the amine group comprises an amino pyridine selected from the group consisting of 2-(methylamino)pyridine, 3-(methylamino)pyridine, 3-dimethylaminopyridine, 3-pyridinemethyldimethyl amine, and combinations thereof.

20. (canceled)

21. The method of claim 1, wherein at least one of the first molecule or second molecule has a molecular weight of more than 200 grams per mole.

22. The method of claim 1, wherein the first molecule has a molecular weight of more than 200 grams per mole.

23. The method of claim 1, wherein the first molecule is a drug selected from the group consisting of anti-inflammatory drugs, anti-cholesterol drugs, ritonavir, carbamazepine, cimetidine, propranolol, atenolol, labetalol, metoprolol, acebutolol, bezafibrate, naproxen, mefenamic acid, diclofenac, acetazolamide, flurosemide, aceclofenac, vitamin B3, a beta blocker, chiral compounds, derivatives thereof, and combinations thereof.

24-26. (canceled)

27. The method of claim 1, wherein the co-crystallized composition is in neutral form.

28. The method of claim 1, wherein the co-crystallized composition comprises ionic co-crystals of the first molecule and the second molecule.

29. The method of claim 1, wherein the first molecule of the co-crystallized composition is in anionic form, and wherein the second molecule of the co-crystallized composition is in cationic form.

30. The method of claim 1, further comprising a step of utilizing the co-crystallized composition as seed crystals to grow additional co-crystallized compositions from the first molecule and the second molecule, wherein the growing comprises placing the co-crystallized composition in a solvent comprising a mixture of the first molecule and the second molecule and evaporating the solvent.

31-51. (canceled)
